(19) United States
(12) Patent Application Publication
(10) Pub. No.: US 2008/0070991 A1
(43) Pub. Date: Mar. 20, 2008

(54) ANIMAL PRODUCT ENRICHMENT USING RESVERATROL

(76) Inventors: Charles H. Cella, Pembroke, MA (US); Brent D. Bliven, Hanover, MA (US)

Correspondence Address: STRATEGIC PATENTS P.C.., C/O PORTFOLIOIP, P.O. BOX 52050, MINNEAPOLIS, MN, 55402, US

(21) Appl. No.: 11/691,360

(22) Filed: Mar. 26, 2007

Related U.S. Application Data
(60) Provisional application No. 60/785,417, filed on Mar. 24, 2006.

(51) Int. Cl. A61K 31/05; A23K 1/00

(52) U.S. Cl. 514/733; 426/635

(57) ABSTRACT

The methods and systems disclosed herein include combining resveratrol with a bioavailability promoter to produce a bioavailable resveratrol, combining the bioavailable resveratrol with a combination compound to produce a bioavailable resveratrol enriched compound, and combining the bioavailable resveratrol enriched compound with an animal delivery method to produce a resveratrol enriched product for use by an animal.

### CROSS-REFERENCE TO RELATED APPLICATIONS

This application claims the benefit of the following commonly-owned U.S. Provisional Patent Application, which is incorporated herein by reference in its entirety: App. No. 60/785,417 filed on Mar. 24, 2006 and entitled “RESVERATROL.”

### FIELD OF THE INVENTION

This disclosure relates to the field of commercial, domesticated, and exotic animal products and more particularly to animal products enriched with resveratrol, including in combination with a bioavailability promoter.

### BACKGROUND

Resveratrol (3,4′,5-trihydroxystilbene and 3,4′,5-stilbenetriol) has been found in numerous in vitro and in vivo studies to have a wide range of beneficial health effects. Among these, resveratrol has been found to protect against atherosclerosis, hyperlipidemia, and other markers of cardiovascular disease, inhibit cancer cell proliferation, protect against osteoarthritis, and have anti-aging effects. The positive effects of resveratrol have been found in both human studies and animal models. However, although resveratrol is a naturally occurring compound, it's presence in plant derivatives is low, and it is absorbed slowly and eliminated quickly. Thus, to obtain the positive health effects of resveratrol, typically long-term and/or high levels of consumption are necessary. Further compounding the problem for commercial and domesticated animals is the fact that many of the plants from which resveratrol may be extracted (e.g., grape skins) are not included in their diets. A need exists for new techniques and products for providing resveratrol to the animal population and improving the bioavailability of resveratrol for animals.

### SUMMARY

Provided herein are methods and systems for combining resveratrol with a bioavailability promoter to produce a bioavailable resveratrol, combining the bioavailable resveratrol with a combination compound to produce a bioavailable resveratrol enriched compound, and combining the bioavailable resveratrol enriched compound with an animal delivery method to produce a resveratrol enriched product for use by an animal.

In embodiments, resveratrol may be organic or synthetic resveratrol. Organic resveratrol may be derived from grapes, berries, pine, Japanese Knotweed, Giant Knotweed, green teas, black tea, Polygonnum cuspidatum, or some other organic source.

In embodiments, resveratrol may be obtained based at least in part on an artificial production stimulus. Artificial stimuli may include Botrytis cinerea, aluminum chloride, aluminum sulfate, ultraviolet-B, ultraviolet-C, stilbene synthase, or some other production stimulus.

In embodiments, the resveratrol may be genetically engineered.

In embodiments, the resveratrol may a plurality of organic resveratrol types.

In embodiments, the resveratrol may a plurality of synthetic resveratrol types.

In embodiments, the resveratrol may a plurality of genetically engineered resveratrol types.

In embodiments, the resveratrol may a combination of organic resveratrol and synthetic resveratrol types.

In embodiments, the resveratrol may a combination of organic resveratrol and genetically engineered resveratrol types.

In embodiments, the resveratrol may a combination of synthetic resveratrol and genetically engineered resveratrol types.

In embodiments, the resveratrol may a combination of organic resveratrol, synthetic resveratrol, and genetically engineered resveratrol types.

In embodiments, the resveratrol may a combination of organic resveratrol and genetically engineered resveratrol types.

In embodiments, the resveratrol may be in the form of micronized particles.

In embodiments, the bioavailability promoter may be a bioadhesive polymer, a microparticle, a nanoparticle, a cationic polymer, a polymeric microsphere, microencapsulation, nanoencapsulation, a structural modification, a plurality of bioavailability promoters, or some other bioavailability promoter or bioavailability promoter combination.

In embodiments, the bioavailability promoter is at least one selected from the group consisting of polyanhydrides, and polymers and copolymers of acrylic acid, methacrylic acid, and their lower alkyl esters, for example polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In embodiments, a cationic polymer may be chitosan, albumin, gelatin, starch, DEAE-Cellulose, cationic guar and DEAE-Dextran. DEAE-Dextran and cationic guar have tertiary amino groups. Cationic guar's INCI name is Guar hydroxypropyltrimonium chloride and DEAE-Dextran is Diethylaminoethyl-Dextran, or some other cationic polymer.

In embodiments, a bioadhesive polymer is poly(fumaric-co-sebacic)anhydride p(FA-:SA), poly(lactic-co-glycolic acid) (PLGA), PLL-g-PEG, a low molecular weight anhydride oligomer, polycaprolactone, non-biodegradable, biodegradable, non-erodible, or some other bioadhesive polymer.

In embodiments, a structural modification may be a nanotechnology, a microparticle, a structure-based digestive delay, or some other structural modification.

In embodiments, a bioavailable resveratrol may be a biologically engineered resveratrol.

In embodiments, a combination compound may be a vitamin, mineral, antioxidant, polyphenol, anti-inflammatory, veterinary drug, or some other compound.

In embodiments, the animal delivery method may be a food stuff, edible toy, semi-edible toy, topical application, liquid, injection, subcutaneous delivery, or some other delivery method.

In embodiments, a resveratrol enriched product for use by an animal may include animal feeds, pet feed, commercial animal feed, dry feed, canned feed, water additive, dog biscuits, gourmet pet feeds, animal strips (e.g., rawhide-type), beef jerky, pepperoni, edible pet toys, bones, animal bones covered/injected with resveratrol, nylon bones with resveratrol, catnip, veterinary medicines, eye drops, ear drops, tongue drops, nasal spray, pill, tablet, capsule, heartworm medication, insulin, tapeworm medicine, vitamins, minerals, dietary supplements, vaccines (e.g., distemper, leukemia, rabies, toxoplasmosis), anti-arthritis/pain meds, carprofen, deracoxib, glucosamine, firocoxib, aspirin, flea/tick medicine (e.g., collar, skin drops, powder), grooming products (e.g., shampoos and soaps), skin care products, intravenous drip, time release patch, topical patch, skin drops, subcutaneous slow-release capsule, or some other product.

In embodiments, the resveratrol enriched product may include a high does resveratrol. A high dose resveratrol may include a dose equal to the sirtuin activating effect of 18, 25, 50, 100, 250, 500, 750 mg/kg resveratrol, 1, 2, 5 g/kg resveratrol, or some other level of high resveratrol dosage.

In embodiments, a high does of resveratrol may be delivered in a sustained release formulation.

In embodiments, an animal may be commercial animals, such as, livestock, cattle, poultry, foul, cows, horses, pigs, bison, buffalo, oxen, sheep, goats, chicken, ducks, pheasant, partridge, emus, llama, ostrich, deer, mink, crocodile, alligator; domestic animals, such as, dog, cat, hamster, fish, gerbil; and exotic animals, birds, rodents, chinchilla, degu, prairie dog, sugar glider, rat, mice, skunk, hedgehog, squirrel, wallaby, iguana, lizard, kimono dragon, ferret, rabbit, hare, guinea pig, possum, fox, pot belly pig, tropical fish, shark, snake, turtle, other reptiles, spiders, or some other animal.

These and other systems, methods, objects, features, and advantages of the present invention will be apparent to those skilled in the art from the following detailed description of the preferred embodiment and the drawings.

### BRIEF DESCRIPTION OF THE FIGURES

The invention and the following detailed description of certain embodiments thereof may be understood by reference to the following figure:

FIG. 1 illustrates a method for making a bioavailable resveratrol enriched animal product.

### DETAILED DESCRIPTION

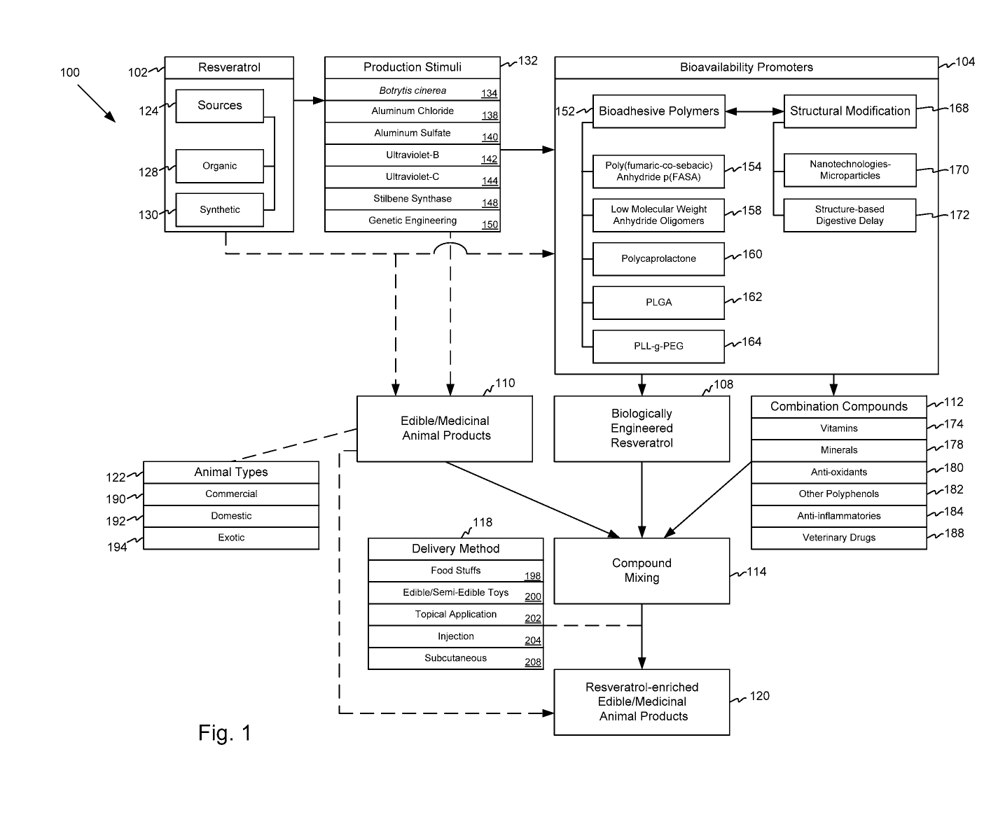

The methods and systems disclosed herein relate to the inclusion of resveratrol in products for commercial, domesticated, and exotic animals, and methods for improving the bioavailability of the resveratrol included in animal products. FIG. 1 summarizes a resveratrol infusion methodology 100 for incorporating resveratrol 102 into products, foodstuffs, and pharmacological agents for animals. Resveratrol 102 (3,4′,5-trihydroxystilbene and 3,4′,5-stilbenetriol) is an organic 128 polyphenol commonly found in the skins of grapes (e.g., the Vitis vinifera variety), the peanut, berries (e.g., within the vaccinur species species), pines, knotweed, green and black tea, and Polygonnum cuspidatur.

In embodiments, the terms “sirtuin activator” or “sirtuin activating compound” may refer to a compound, such as resveratrol, that increases the level of a sirtuin protein and/or increases at least one activity of a sirtuin protein. In an embodiment, a sirtuin activator may increase at least one biological activity of a sirtuin protein by at least about 10%, 25%, 50%, 75%, 100%, or more. Exemplary biological activities of sirtuin proteins may include deacetylation, e.g., of histones; extending lifespan; increasing genomic stability; silencing transcription; and controlling the segregation of oxidized proteins between mother and daughter cells, or some other biological activity.

Resveratrol 102 has been found to protect against atherosclerosis, hyperlipidemia, and other markers of cardiovascular disease, inhibit cancer cell proliferation, protect against osteoarthritis and osteoporosis, have anti-aging effects, anti-inflammatory effects, to speed the clearance of amyloid-beta peptides that are thought to be associated with Alzheimer's Disease, assist the functioning of the pancreas, and many others. The beneficial health effects of resveratrol 102 have been attributed to its antioxidant properties, its anticyclooxygenase activity, and its ability to improve lipid and lipoprotein metabolism. Resveratrol 102 also inhibits platelet aggregation and exhibits antiestrogenic activity. The proliferation of various human malignant cell lines is slowed by resveratrol 102. The inhibition of malignant cell growth that is associated with resveratrol 102 is accompanied by the accumulation of cells in S and G₂ phases. The effects on cell cycle progression may also be explained by the direct inhibition of ribonucleotide reductase and DNA polymerase. The number and position of the hydroxylic groups in the resveratrol molecule have been suggested to play an important role in the antioxidant activity.

Resveratrol 102 may be obtained through organic 128 or synthetic 130 means. Resveratrol 102 is produced in plants, in part, as a defense against fungus, environmental stress, or in response to a pathogenic attack. In particular, the fungus Botrytis cinerea 134, and two metabolites of Botrytis cinerea 134, alginate and mucic acid, have been found to be potent inducers of resveratrol 102 production. Environmental levels of Botrytis cinerea 134 are generally higher in colder climates. Grapes (and other plants) from colder climates often produce higher concentrations of resveratrol 102 than similar species growing in warmer climates. Thus, manipulation of the level of the Botrytis cinerea 134 fungus in a plant's environment may increase the resveratrol 102 levels of plants. However, studies have found that once the plants produce enough resveratrol 102 to optimize their immunity to the fungus for self preservation, resveratrol 102 production plateaus.

Resveratrol 102 may occur in the form of trans-resveratrol or cis-resveratrol. Trans-resveratrol is more strongly associated with beneficial health effects than is cis-resveratrol. One molecule of trans-resveratrol may be synthesized from one molecule of p-coumaroyl-CoA and three molecules of malonyl-CoA. Various methods may be used to artificially increase the production of resveratrol 102 in plants, including the use of stilbene synthase 148. Artificially treating grape plants with aluminum chloride 138 or aluminum sulfate 140 has been found to increase resveratrol 102 production, as has irradiating resveratrol 102 producing plants with ultraviolet B 142 and ultraviolet C 144 light.

Methyljasmonate has been shown to be effective an effective synthetic 130 stimulator of both trans- and cis-resveratrol endogenous accumulation, as well as resveratrol 102 release into a culture medium. Similarly, jasmonic acid has been found to promote endogenous resveratrol 102 accumulation, but less markedly than methyljasmonate. Na-orthovanadate has been shown to promote the production and/or accumulation of cis-resveratrol, but not trans-resveratrol levels. Significantly, experimental evidence exists for ex-novo synthesis of resveratrol using methyljasmonate. For example, gel analysis was performed by Tassoni (Tassoni, A., et al, Jasmonates and Na-orthovanadate promote resveratrol production in Vitis vinifera cv. Barbera cell cultures. New Phytologist. 2005; 166 (3), incorporated by reference herein) on Barbera cell suspensions of the cultivar, Vitis vinifera cv, treated with 10 μm of methyljasmonate. The results demonstrated an up-regulation of the stilbene synthase 148, evidence that the methyljasmonate stimulated the resveratrol 102 synthesis ex-novo of the protein.

In embodiments, genetic engineering 150 may be used to manufacture higher quantities of resveratrol 102 naturally found and to manufacture resveratrol in plant varieties where it is not normally produced. For example, resveratrol 102 synthase genes have been isolated, cloned and inserted into potatoes, rice, tomatoes, alfalfa and tobacco.

In embodiments, resveratrol enriched animal products may be combined with antioxidants in order to minimize the destructive oxidation process which may negatively impact the effectiveness of resveratrol. Oxidation in animal products, such as feeds, is associated with a loss of nutrient quality. During oxidation, heat may be released thereby causing degradation of an animal product. Oxidation may damage fat soluble vitamins, decrease the protein value of the product, decrease the energy store of the product, and or degrade heat labile protein. Oxidation may also increase the formation of free radicals which have been found to negatively impact animals' immune systems, longevity, and product shelf life. The reaction sequence for oxidation can be divided into distinct phases. An initiation phase of the oxidation reaction can be catalyzed by a number of factors including light, metals, surface area, and or exposure to oxygen and temperature. This reaction may be halted with the addition of antioxidants. Antioxidants act as free radical “scavengers,” terminating the propagation sequence, and dissipating the reactive energy across their ringed structure. Fat is an ingredient that is highly susceptible to oxidation as a result of its chemical structure. Oxidation occurs most readily in fats with multiple points of unsaturation (double bonds) in their fatty acid chains. Commonly used fats in animal products, to name but several, include animal fat, poultry fat, animal tallow, animal-vegetable fat blend, and canola oil, all of which are susceptible to degradation by oxidation. Other common animal product ingredients that are susceptible to oxidation include fish meal, fat-soluble vitamins (A, D, E and K) and natural pigments. Antioxidants may limit oxidation in animal products incorporating fats, poultry or fish meal, preserve the vitamin quality, as well as preserve the potential for pigmentation of the product to improve its salability.

By combining resveratrol with an antioxidant or antioxidant blend in an animal product, the resulting product may have an improved effectiveness for supporting animal health by both preserving the potency of the resveratrol as well as minimizing the degradation of other animal product vitamins and nutrients. A resveratrol enriched animal product that is combined with an antioxidant or antioxidant blend may also have a greater shelf life. A lengthened shelf life may improve logistic and financial efficiencies by permitting longer shipping distances, thereby permitting centralized production, storage in distribution centers, packaging in larger quantities, larger retail inventories, longer consumer use from a single purchased unit, and the like.

Synthetic and natural antioxidants may be combined with an animal product, such as a resveratrol enriched product, in order to prevent oxidation of the product. One example, selected from several examples, of a natural antioxidant that may be used to prevent oxidation in a resveratrol enriched product is vitamin E. Vitamin E may be used as an antioxidant in the form of alpha-tocopherol, or in the form of mixed tocopherols. Alpha-tocopherol is the biologically active form of vitamin E, but has reduced antioxidant efficacy relative to the delta and gamma forms. The delta and gamma forms generally have greater antioxidant effects; however these forms may be less nutritional. Other sources of natural antioxidants include mixed tocopherols, such as cranberries, blueberries, apples, and other fruits, vitamin C, citric acid and rosemary.

Synthetic antioxidants are generally more effective than natural antioxidants, such as vitamin E. Synthetic antioxidants may also be less expensive to produce than natural antioxidants. Part of the superiority of synthetic antioxidants derives from the fact that they are more stable, and show better survivability when added during the rendering process and through feed processing and pelleting of an animal product, such as a feed.

The incorporation of antioxidants into an animal product may occur in stages and be blended with the incorporation of other animal product ingredients, including resveratrol. Antioxidants may be added to an animal product in a rendering facility. Because the oxidation process involves a rapid reaction, the antioxidant may be added as early in the rendering process as is physically practical. This may be accomplished in the rendering facility within the raw offal stream as the material enters the rendering plant cookers. Fat-soluble antioxidants, such as ethoxyquin, may be proportionately distributed in the liquid fat and fat portion of by-product meal after pressing. Adding an antioxidant like ethoxyquin to the raw offal may adequately stabilize liquid fat. The antioxidant incorporation resulting from such a process with liquid fat may produce protective antioxidant levels. Following the initial incorporation of antioxidants with the animal fat, other materials for the end product may be added prior to and or after the cooking of the product (if cooking is necessary) including resveratrol, vitamins, flavorings, colorings, and the like. Following this, the animal product may be treated one or more additional times with an antioxidant for additional oxidation protection of the final product, including protection of the incorporated resveratrol

The beneficial effects of resveratrol in a resveratrol enriched animal product may be increased by improving the bioavailability of the resveratrol contained within the animal product. Bioavailability refers to the extent that a compound is available for use by the animal ingesting or coming into contact with that compound. Bioadhesives (e.g., bioadhesive polymers) have been found to improve the bioavailability of numerous compounds based at least in part on prolonging the contact time of a compound with a body tissue and enabling a more controlled release of the compound to the tissue.

In embodiments, organic 128 or synthetically 130 produced resveratrol 102 may be combined with bioadhesives 152 to improve the bioavailability 104 of the resveratrol 102. Numerous studies have found that absorption of resveratrol 102 in animal models and humans is low. Without adequate bioavailability 104, the beneficial health effects of resveratrol 102 may be minimized. For example, Walle (Walle, T. High absorption but very low bioavailability of oral resveratrol in humans, Drug Metabolism and Disposition, 2004 (32) 1377-1382, incorporated by reference herein) examined the absorption, bioavailability 104, and metabolism of resveratrol 102 after oral doses in humans. The absorption of a 25-mg oral dose was 70%, with peak plasma levels of resveratrol 102 and metabolites of 491±90 ng/ml (about 2 μM) and a plasma half-life of 9.2±0.6 h. Only trace amounts of unchanged resveratrol 102 (<5 ng/ml) were detected in plasma. Most of the oral dose was recovered in urine. Three metabolic pathways were identified: sulfate and glucuronic acid conjugation of the phenolic groups and, hydrogenation of the aliphatic double bond. The authors concluded that extremely rapid sulfate conjugation by the intestine and liver were the primary rate-limiting step in resveratrol's 102 bioavailability 104. Although the bioavailability 104 of resveratrol 102 was very low, accumulation of resveratrol 102 was found in the epithelial cells along the aerodigestive tract.

In embodiments, bioadhesive polymers 152 may be used in conjunction with resveratrol 102 to improve to improve its bioavailability 104 by providing additional contact with the digestive tract of an animal ingesting a resveratrol enriched product. Biologically adhesive delivery systems may have advantages over conventional drug delivery systems. Polymers 152 may be engineered to have specific properties that promote strong adhesion to the gastrointestinal mucus and cellular linings. For example, Mathiowitz (Mathiowitz, E. Biologically erodable microspheres as potential oral drug delivery systems. Nature. 1997 Mar. 27;386(6623):410-4., incorporated by reference herein) report that engineered polymer microspheres 170 made of biologically erodable polymers 152 may traverse both the mucosal absorptive epithelium and maintain contact with intestinal epithelium for extended periods of time and actually penetrate it, through and between cells. Thus, once such a bioadhesive polymer 152 microsphere 170 is loaded with resveratrol 102, the microspheres 170 may be developed as delivery systems to transfer resveratrol 102 molecules to the animal's circulatory system. The clinical efficacy of the bioadhesive 152 delivery model has been demonstrated in improving the bioavailability 104 of compounds of diverse molecular size, such as, dicumarol, insulin, and plasmid DNA. Such a resveratrol-bioadhesive combination may, in turn, be further incorporated with other ingredients comprising the resveratrol enriched animal end product (e.g., animal products, vitamins, flavorings, colorings, etc.).

In embodiments, resveratrol may be combined with a cationic polymer prior to inclusion in an animal product. In embodiments, resveratrol may be combined with a plurality of cationic polymers prior to inclusion in an animal product. A cationic polymer may include a component of the resveratrol delivery system that assists in the release of the active resveratrol agent.

In embodiments, cationic polymers suitable for combination with resveratrol may include, but is not limited to, those polymers having one positive charge per 100 amu to 2000 amu. Examples of such polymers include albumin, gelatin, starch, DEAE-Cellulose, cationic guar and DEAE-Dextran. DEAE-Dextran and cationic guar have tertiary amino groups. Cationic guar's INCI name is Guar hydroxypropyltrimonium chloride and DEAE-Dextran is Diethylaminoethyl-Dextran.

In embodiments, resveratrol may be combined with a high viscosity cationic polymer, such as chitosan, prior to inclusion in an animal product. Chitosan may a molecular weight of at least about 100,000 Daltons, more preferably at least about 250,000 Daltons and most preferably at least about 300,000 Daltons. Chitosan is a natural, biodegradable cationic polysaccharide derived by deacetylating chitin, a natural material extracted from fungi, the exoskeletons of shellfish and from algae. Chitosan comprises a family of polymers with a high percentage of glucosamine (typically 70-99%) and N-acetylated glucosamine (typically 1-30%) forming a linear saccharide chain of molecular weight from 10,000 up to about 1,000,000 Dalton. Typically, chitosan used in the invention is 70-100% glucosamine, such as 70-90% glucosamine or 80-100% glucosamine, more typically 85-95% glucosamine. Chitosan, through its cationic glucosamine groups, interacts with anionic proteins such as keratin in the skin conferring some bioadhesive characteristics.

In embodiments, resveratrol may be combined with micron and sub-micron polymeric microspheres prior to inclusion in an animal product. In an example, phase inversion nanoencapsulation may be used in which a polymer is dissolved in a solvent and the resveratrol to be encapsulated is dissolved or suspended in the polymer solution. The resulting solution or suspension may be diluted with a solution that is a non-solvent for the polymer, such as an animal food product. The non-solvent may be selected to be sufficiently miscible with the solvent so that a single-phase solution that is a non-solvent for the polymer is formed after the dilution. The spontaneous mixing of the two solutions may occur rapidly and with a small characteristic scale of mixing. As a result, the polymer may precipitate to form particles with a very small diameter, typically in the range of tens to hundreds of nanometers, or in some cases up to several microns in diameter. These particles may be generally uniform in size. The resveratrol may be encapsulated in the nanospheres. Upon administration to an animal, the resveratrol may be released from the nanospheres by diffusion, degradation of the polymer, or a combination of these effects.

In embodiments, resveratrol may be combined with bioadhesive polymers that target mucosal surfaces, as in the gastrointestinal tract, including polymers, such as polyanhydrides, and polymers and copolymers of acrylic acid, methacrylic acid, and their lower alkyl esters, for example polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In embodiments, non-biodegradable or biodegradable polymers may be used to encapsulate the resveratrol. Non-biodegradable polymers may include ethylene vinyl acetate, poly(meth)acrylic acid, polyamides, copolymers and mixtures thereof. Biodegradable polymers may include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

In embodiments, non-erodible polymers may be used for oral administration of a resveratrol enriched animal product.

In embodiments, resveratrol may be combined with natural polymers, including natural biopolymers that degrade by hydrolysis, such as polyhydroxybutyrate. Natural polymers may include proteins such as albumin, collagen, gelatin and prolamines, for example, zein, and polysaccharides such as alginate, cellulose derivatives and polyhydroxyalkanoates, for example, polyhydroxybutyrate.

In embodiments, resveratrol may be combined with synthetic polymers. Synthetic polymers may include: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone)-, copolymers and blends thereof. As used herein, “derivatives” may include polymers having substitutions, additions of chemical groups and other modifications routinely made by those skilled in the art.

In embodiments, bioadhesive polymers 152 may be combined with structural modification 168, such as, nanotechnologies 170 that may reduce the particle size of resveratrol 102, thereby increasing its bioavailability 104 within animals. Experimental evidence has shown that both particle size and polymer 152 composition are important factors affecting the bioavailability 104 of pharmaceutically active compounds. For example, Thanos (Thanos, C. G., Enhancing the oral bioavailability of the poorly soluble drug dicumarol with a bioadhesive polymer. J Pharm Sci. 2003 August;92(8):1677-89., incorporated by reference herein) reduced the particle size of the drug, dicumarol, and combined it with varying sizes of bioadhesive 152 microspheres 170 composed of poly(fumaric-co-sebacic) anhydride p(FA:SA) 154. Swine were catheterized and gavaged, and blood collected for pharmacokinetic analysis. Smaller drug particles showed the highest bioavailability: micronized drug with 7% p(FA:SA) 17:83 polymer 152 had 190% relative bioavailability, and phase inverted p(FA:SA) 154 17:83 microspheres with 31% (w/w) loading had 198% relative bioavailability to spray dried formulation. Formulations with larger drug particles achieved 71% relative bioavailability. A nonadhesive formulation, fabricated with poly(lactic acid) (PLA), showed 91% relative bioavailability. Thus, both particle size 170 and polymer 152 composition play a role in bioavailability and may be used to increase absorption of resveratrol in animals.

In embodiments, resveratrol may be further combined with nanoparticles prior to inclusion in an animal product. Nanoparticles refers to particles that are smaller than microparticles. Microparticles have a mean diameter of 1 micron to 100 microns, such as from 1 micron to 50 microns, 1 micron to 20 microns or 1 micron to 10 microns. Typically, nanoparticles have a mean diameter of less than 1 micron or less than 500 nm, such as from 20 nm to 500 nm, from 20 nm to 300 nm, from 50 nm to 200 nm or from 50 nm to 150 nm. Preferably, greater than 90%, greater than 95%, greater than 97%, greater than 98% or greater than 99% of the particles fall within one of these ranges. Preferably, particle uniformity is such that particles in a group having a particular mean diameter are have individual diameters that are within 50% of the mean diameter, such as within 25% or even within 10%.

In embodiments, resveratrol 102 may be combined with anhydride oligomers within polymer microsphere blends. Santos (Santos, C. A., et al, Evaluation of anhydride oligomers within polymer microsphere blends and their impact on bioadhesion and drug delivery in vitro. Biomaterials. 2003 September;24(20):3571-83., incorporated by reference herein) found that resveratrol 102 may be combined with blends of low molecular weight anhydride oligomers 158 with thermoplastic poly(fumaric-co-sebacic anhydride) [p(FASA)] 154 and polycaprolactone 160 to improve bioadhesion and the bioavailability of resveratrol.

In embodiments, resveratrol 102 may be combined with poly(lactic-co-glycolic acid) (PLGA) 162 and P(FASA) 154 plasmid-loaded nanospheres 170 for drug delivery. (Sandor, M., et al, Transfection of HEK cells via DNA-loaded PLGA and P(FASA) nanospheres. J Drug Target. 2002 September;10(6):497-506., incorporated by reference herein).

In embodiments, resveratrol 102 may be combined with poly(L-lysine)-g-poly(ethylene glycol) (PLL-g-PEG) 164, to produce PLL-g-PEG-coated 164 PLGA 162 microspheres 170 for drug delivery. (Muller, M. Surface modification of PLGA microspheres. J Biomed Mater Res A. 2003 Jul. 1;66(1):55-61, incorporated by reference herein). Resveratrol 102 may also be delivered in a physically modified structure 168 that mechanically delays 172 passage through an animals digestive system, thereby increasing the time for resveratrol 102 bioavailability 104.

Following the combination of resveratrol 102 and other bioavilability promoters 104 including, but not limited to, those bioadhesive polymers 152 and/or structurally modified 168 particles described herein, a biologically engineered resveratrol 108 may be available for further compound mixing 114. For example, the biologically engineered reseveratrol 1028 may be further mixed with compounds, such as, vitamins 174, minerals 178, anti-oxidants 180, other polyphenols 182, anti-inflammatories 184, and/or other veterinary drugs 188. These combination compounds 112 may also be combined with similar bioavailability promoters 104 as used with the resveratrol 102.

In embodiments, micronized particles of resveratrol may be microencapsulated in one or more polymers, for example, using standard microencapsulation and nanoencapsulation techniques. The micronized particles resveratrol, for example formed by precipitation in alcohol, may serve as a core material in many standard encapsulation processes. The core material typically may encapsulated in a polymeric material. Common microencapsulation techniques include interfacial polycondensation, spray drying, hot melt microencapsulation, and phase separation techniques (solvent removal and solvent evaporation).

In embodiments, there may be multiple delivery methods 118 for making the resveratrol 102 and combination compounds 112 available to animals within the resveratrol-enriched product 120. For example, the compounds may be mixed with foodstuffs 198, with edible or semi-edible toys 200, made available within a topical application (e.g., a skin creme, or biodelivery patch) 202, produced in injection form 204, and/or for subcutaneous application 208. Alternatively, the resveratrol 102 may be directly incorporated into edible/medicinal animal products 110 in order to derive resveratrol-enriched edible/medicinal animal products 120.

In embodiments, compound mixing 114 to produce a resveratrol enriched edible/medicinal animal product 120 may include resveratrol 102, biologically engineered resveratrol 108, and or combination compounds 112 then, in turn, combined with a delivery method 118. Delivery methods may include, but are not limited to, food stuffs, edible toys, semi-edible toys, topical applications, injections, subcutaneous delivery means, and the like. For example, this compound mixing may be, to cite only one example, biologically engineered resveratrol 108 that is bound to a veterinary drug 188 both of which have undergone structural modification 168 to reduce the resveratrol 102 and the drug 188 to microparticles 170, with the resulting combination encapsulated within a bioadhesive polymer 154 for improved bioavailability 104. Once the compound mixing 114 is completed, the resulting compound may be mixed, incorporated, or bound with a delivery method 118 to produce a resveratrol enriched edible/medicinal animal product 120.

In embodiments, “sirtuin activating effect” may refers to the level or extent of one or more therapeutic effects obtained upon administration of a high dose of reservatrol. In embodiments, a high dose of a resveratrol may refer to an amount having a sirtuin activating effect equal to or greater than the sirtuin activating effect of 18 mg/kg resveratrol.

In embodiments, a high dose of a resveratrol may refer to a quantity having a sirtuin activating effect equal to or greater than the sirtuin activating effect of 18 mg/kg of resveratrol which is administered (i) orally, (ii) released from a sustained release form over 6 to 48 hours, and/or (iii) for an equivalent amount of time.

In embodiments, a high dose of a resveratrol may refer to a quantity having a sirtuin activating effect equal to or greater than the sirtuin activating effect of at least about 20, 25, 30, 35, 40, 50, 60, 75, 100, 150 mg/kg, or more, or resveratrol.

In embodiments, a high dose may be administered to an animal once, or multiple times (e.g., daily).

In embodiments, a high dose may be administered to an animal until a desired therapeutic effect is achieved. For example, a high dose may be administered daily for 1 day, 1 week, 2 weeks, 1 month, 2 months, 3 months, 6 months, 1 year, or more depending on the disease or disorder being treated.

In embodiments, a high dose of a sirtuin activator, such as resveratrol, may be administered daily in a single dosage or may be divided into multiple dosages (e.g., dosages taken twice or three times per day).

In embodiments, a high dose of a sirtuin activator, such as resveratrol, may be administered in a sustained release formulation.

In embodiments, a high dose of a sirtuin activator, such as resveratrol, may be taken alone or in combination with other compounds.

In embodiments, a mixture of a high dose of two or more sirtuin activating compounds, including resveratrol, may be administered to an animal.

In embodiments, a high dose of a sirtuin activator, such as resveratrol, may be administered in response to a special therapeutic need in an animal.

In embodiments, a high dose of a sirtuin activator, such as resveratrol, may be administered with other compounds, including, but not limited to butein, Fisetin, piceatannol, or quercetin.

In embodiments, a high dose of one or more sirtuin activating compounds, including resveratrol, may be administered with one or more therapeutic agents for the treatment or prevention of various animal diseases

In embodiments, combination therapies comprising a high dose of a sirtuin activating compound, such as resveratrol, may refer to (i) pharmaceutical compositions that comprise a high dose of one or more sirtuin activating compounds in combination with one or or more therapeutic agents, and (ii) co-administration of a high dose of one or more sirtuin activating compounds with one or more therapeutic agents wherein the sirtuin activating compound and therapeutic agent have not been formulated in the same compositions. When using separate formulations, the high dose of the sirtuin activating compound may be administered at the same, intermittent, staggered, prior to, subsequent to, or combinations thereof, with the administration of another therapeutic agent.

In embodiments, the high dose of a sirtuin activating compound, such as resveratrol, may be administered in a sustained release formulation (e.g., by embedding or encapsulating the sirtuin activator into nanoparticles for delivery over a period of at least 12 hours). In embodiments where resveratrol is administered to an animal in a sustained release formulation, a high dose of the resveratrol may be administered for sustained delivery over a period of for example, at least about 12, 15, 18, 24, or 36 hours, or longer.

In embodiments the resveratrol may be administered for a sustained delivery over a period of one or more days.

In embodiments, resveratrol may be administered for a sustained delivery over a period of one or more weeks.

In embodiments, the sirtuin activating compound, such as resveratrol, animal food or liquid may contain from about 0.1% to about 99%, or from about 0.1% to about 10% of a sirtuin activator by weight. In embodiments, a high dose as described herein of resveratrol may be administered in a single serving of an animal food, or liquid.

In emboidments, a single dosage form may be provided, for example a 10 fluid ounce serving of a liquid such as water, flavored water, or the like that contains a quantity of resveratrol equal to or greater than 25 mg.

In embodiments, a single dosage form may be provided that contains a quantity of resveratrol of about 10, 15, 20, 25, 50, 60, 75, 80, 100, 150, 200, or more, mg resveratrol per 8 fluid ounces.

In embodiments, a single dosage form may be provided (e.g., a serving of animal food such as dried animal meal) that contains a total quantity of 100 mg resveratrol.

In embodiments, a single dosage form may be provided that contains a quantity of resveratrol of about 10, 15, 20, 25, 50, 60, 75, 80, 100, 150, 200, or more, mg resveratrol per 100 to 500 kcal.

In embodiments, the resveratrol enriched edible/medicinal animal product may be one selected from the group of animal feeds, pet feed, commercial animal feed, dry feed, canned feed, water additive, dog biscuits, gourmet pet feeds, animal strips (e.g., rawhide-type), beef jerky, pepperoni, edible pet toys, bones, animal bones covered/injected with resveratrol, nylon bones with resveratrol, catnip, veterinary medicines, eye drops, ear drops, tongue drops, nasal spray, pill, tablet, capsule, heartworm medication, insulin, tapeworm medicine, vitamins, minerals, dietary supplements, vaccines (e.g., distemper, leukemia, rabies, toxoplasmosis), anti-arthritis/pain meds, carprofen, deracoxib, glucosamine, firocoxib, aspirin, flea/tick medicine (e.g., collar, skin drops, powder), grooming products (e.g., shampoos and soaps), skin care products, intravenous drip, time release patch, topical patch, skin drops, subcutaneous slow-release capsule, and the like.

Animal types 122 that may benefit from resveratrol-enriched animal products 120 include commercial animals 190, such as, livestock, cattle, poultry, foul, cows, horses, pigs, bison, buffalo, oxen, sheep, goats, chicken, ducks, pheasant, partridge, emus, llama, ostrich, deer, mink, crocodile, alligator; domestic animals 192, such as, dog, cat, hamster, fish, gerbil; and exotic animals 194, birds, rodents, chinchilla, degu, prairie dog, sugar glider, rat, mice, skunk, hedgehog, squirrel, wallaby, iguana, lizard, kimono dragon, ferret, rabbit, hare, guinea pig, possum, fox, pot belly pig, tropical fish, shark, snake, turtle, other reptiles, spiders, and the like.

While the invention has been disclosed in connection with certain preferred embodiments, other embodiments will be recognized by those of ordinary skill in the art, and all such variations, modifications, and substitutions are intended to fall within the scope of this disclosure. Thus, the invention is to be understood with reference to the following claims, which are to be interpreted in the broadest sense allowable by law.

1. A method of producing a resveratrol enriched animal product comprising:
combining resveratrol with a bioavailability promoter to produce a bioavailable resveratrol;
combining the bioavailable resveratrol with a combination compound to produce a bioavailable resveratrol enriched compound; and
combining the bioavailable resveratrol enriched compound with an animal delivery method to produce a resveratrol enriched product for use by an animal.

2. The method of claim 1, wherein the resveratrol is organic resveratrol.

3-15. (canceled)

16. The method of claim 1, wherein the resveratrol is synthetic resveratrol.

17. The method of claim 1, wherein the resveratrol is combined with an artificial production stimulus.

18-23. (canceled)

24. The method of claim 1, wherein the resveratrol is a genetically engineered reseveratrol.

25-28. (canceled)

29. The method of claim 1, wherein the resveratrol is a combination of a synthetic and a genetically engineered resveratrol.

30. (canceled)

31. The method of claim 1, wherein the resveratrol is resveratrol in the form of micronized particles.

32. The method of claim 1, wherein the bioavailability promoter is a bioadhesive polymer.

33-35. (canceled)

36. The method of claim 1, wherein the bioavailability promoter is a microparticle.

37. The method of claim 1, wherein the bioavailability promoter is a nanoparticle.

38-40. (canceled)

41. The method of claim 1, wherein the bioavailability promoter is a polymeric microsphere.

42. The method of claim 1, wherein the bioavailability promoter is microencapsulation.

43. The method of claim 1, wherein the bioavailability promoter is nanoencapsulation.

44-45. (canceled)

46. The method of claim 1, wherein the bioavailability promoter is at least one selected from the group consisting of polyanhydrides, and polymers and copolymers of acrylic acid, methacrylic acid, and their lower alkyl esters, for example polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

47-50. (canceled)

51. The method of claim 1, wherein the bioavailability promoter is a structural modification.

52. The method of claim 51, wherein the structural modification is a nanotechnology.

53-56. (canceled)

57. The method of claim 1, wherein the bioavailable resveratrol is a biologically engineered resveratrol.

58-65. (canceled)

66. The method of claim 1, wherein the animal delivery method is a food stuff.

67-72. (canceled)

73. The method of claim 1, where in the resveratrol enriched product includes a high dose resveratrol.

74-85. (canceled)

86. A system of producing a resveratrol enriched animal product comprising:
a first combination facility adapted to combine resveratrol with a bioavailability promoter to produce a bioavailable resveratrol;
a second combination facility adapted to combine the bioavailable resveratrol with a combination compound to produce a bioavailable resveratrol enriched compound; and
a third combination facility adapted to combine the bioavailable resveratrol enriched compound with an animal delivery system to produce a resveratrol enriched product for use by an animal.

87-170. (canceled)
